(19) United States
(12) Patent Application Publication
(10) Pub. No.: US 2009/0319024 A1
(43) Pub. Date: Dec. 24, 2009

(54) REMOVABLE STENT AND METHOD OF MANUFACTURE THEREOF

(76) Inventors: William B. Long, Gladwyne, PA (US); Ann Marie Joyce, Lexington, MA (US)

Correspondence Address: Pearl Cohen Zedek Latzer, LLP, 1500 Broadway, 12th Floor, New York, NY, 10036, US

(21) Appl. No.: 12/162,983

(22) PCT Filed: Feb. 1, 2007
(86) PCT No.: PCT/US07/02769

§ 371 Date: Mar. 24, 2009

Related U.S. Application Data
(60) Provisional application No. 60/764,770, filed on Feb. 3, 2006.

(51) Int. Cl. A61F 2/06; B26D 3/00

(52) U.S. Cl. 623/1.15; 83/54

(57) ABSTRACT

A removable stent may include, for example, an extension or protrusion, or a cut or angled end, which may be easily grasped for removal by for example forceps. A stent may be molded to form such graspable extensions, or may be cut and/or melted or otherwise deformed as part of manufacture.

### FIELD OF THE INVENTION

The present invention relates to stents and the methods for preparation of stents.

### BACKGROUND OF THE INVENTION

Stents are medical devices that may be inserted into a body, typically via a body cavity, and may be implanted into a lumen or a body cavity passageway, for example, blood vessels or the pancreatic or bile ducts. Stents may be used to maintain or widen a lumen or passageway through a body cavity. Although some stents may remain in the body for extended periods of time, many stents may be used temporarily. When a stent is no longer needed, it may be removed. For example, after a period of time, typically several weeks or months, stents inserted into the pancreatic or bile ducts may become occluded, and may be removed. Removing a stent from a body may involve a stent removal device. For example, a snare or lasso inserted through an endoscope may secure the portion of the stent that protrudes into the intestine, and the stent may be removed via the endoscope channel.

Removing a stent from a body may be difficult or complicated. In the case where a removable stent is a separate entity from the device used to remove the stent, complications may result from, for example, an insecure or poorly positioned connection between the stent and the stent removal device. For example, a snare or lasso may secure the stent some distance from its tip. Securing the stent in this way may cause the stent to fold or double over when it is pulled through the endoscope for removal. Since the diameter of the endoscope may be only slightly larger than the diameter of the stent, the removal of a folded stent may be difficult, or the snare or lasso devices may break, or the channel of the endoscope may be damaged.

It may be difficult to secure an in vivo stent, for example, at its end. A need exists for a stent that may be easily secured at its end for removal.

### SUMMARY OF THE INVENTION

Embodiments of the present invention include a removable stent and a method for manufacture thereof. Such stents may include, for example, extensions or protrusions, or cut or angled ends, which may be easily grasped by for example forceps. A stent may be molded to form such graspable extensions, or may be cut and/or melted or otherwise deformed as part of manufacture.

### BRIEF DESCRIPTION OF THE DRAWINGS

FIG. 1 is a diagram of an exemplary embodiment of a modified stent, including a protrusion;

FIG. 2 is a diagram of an exemplary embodiment of a modified stent, where one edge may form a bulge along a protrusion; and

FIG. 3 is a diagram of an exemplary embodiment of a stent with an attachment.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

A stent according to embodiments of the present invention may be inserted into a body, typically via a body cavity, and may be implanted into any suitable lumen or body cavity passageway, for example, blood vessels or the pancreatic or bile ducts.

A stent according to embodiments of the present invention may include a passageway and two or more openings, with at least one opening at each end that may allow matter such as fluid to flow through the stent. An opening may be the area where material enters or exits the passageway. “Openings” when used herein typically do not include holes that are part of the material of the stent. For example, in a stent according to the present invention made of wire mesh, the holes in the wire mesh may not be considered “openings”. The stent according to some embodiments typically has two ends, but may have more ends. An opening may be located at each end of the stent. The stent may have multiple openings at each end.

In various embodiments, the stent may take any suitable form or design, including, for example, any suitable curvature, protrusions, length or diameter and may be made out of any suitable material, for example, wire, wire mesh, plastic, silicone, or a suitable polymer, for example, biocompatible or medical grade polymer. In some embodiments the stent does not include holes or a mesh in the body of the stent. The stent may be configured as a tube such as a straight tube or as a “t-tube” or a “c-tube”. A tube may refer to a passageway with two or more openings of any suitable design including, for example, any suitable curvature, protrusions, length or diameter.

In some embodiments, during removal from an in-vivo site, the stent is not collapsed or destroyed. A stent according to embodiments of the present invention may be, for example, more efficiently, inexpensively or easily removed.

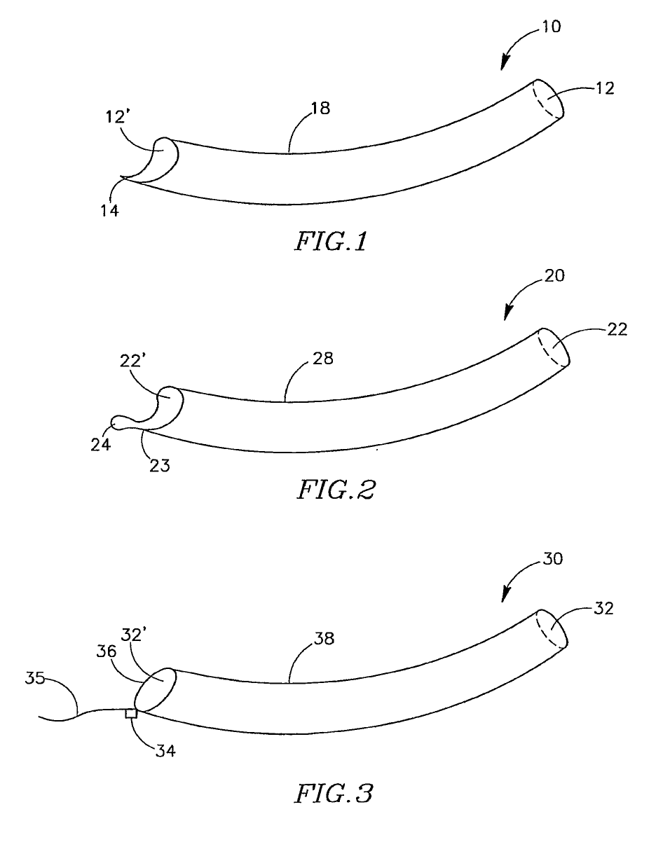

Reference is made to FIG. 1, a diagram of an exemplary embodiment of a stent. Stent 10 may include a tube 18 having openings 12 and 12′. Protruding tip 14 may be part of a cut or angled opening 12′. Stent 10 may be produced by, for example, cutting along the tip or edge of a stent opening to form stent 10 with a protruding tip 14 at its opening 12′. Other methods of producing stent 10 may be used; for example stent 10 may be formed by molding. In one embodiment stent 10 may be cut at an opening such as opening 12′, where the line of a cut or where the tangent line along the curve of a cut is not perpendicular to the axis of the stent 10. The axis of a stent may be any line that runs along the length of the stent, for example, a line, contained in the stent passageway, whose points are equidistant from the points of the stent passageway. The cut may be curved so that a narrower tip is formed, but in other embodiments may be straight.

In another embodiment stent 10 may be cut at an opening, such as opening 12′, such that a section of the cut edge of stent 10 may protrude farther from a perpendicular cross-section of stent 10 than any other section of the cut edge, where the perpendicular cross-section may be taken closest to the end of the cut opening where the cross-section is fully contained by the stent 10 material. In another embodiment a stent may be cut at an opening, such that some portion of the edge 14 of the open end of stent 10 extends further along the axis of stent 10 than the rest of the edge.

Reference is made to FIG. 2, a diagram of a stent according to one embodiment of the invention. Stent 20 may include a tube 28 with openings 22 and 22′. Stent 20 may include a bead, extension, handle or bulge 24 at a protruding tip 23. Bulge 24 of stent 20 may be produced, for example, by melting a stent tip 23 at an opening, for example, by melting tip 14. On melting, the tip 14 or bulge 24 may enlarge or change shape somewhat. Stent 20 may be made of any material that can melt or may be viscous at certain temperatures, for example, plastic, silicone, suitable polymers or various metal materials including wire mesh. In other embodiments, a bulge, bead or handle may be produced by a different method, such as forming in a mold, bending, compression, or by use of appropriate glues, additional hardenable substances, etc.

Reference is made to FIG. 3, a diagram of a stent according to one embodiment of the invention. Stent 30 may include a tube 38 with openings 32 and 32′, and an appendage, attachment or extension 35. Stent 30 and extension 35 in one embodiment are not part of one continuous material, but rather are attached during manufacturing. Any suitable extension 35, for example, a string, wire or hook, may be connected to the stent 30. Stent 30 and extension 35 may be connected by a connection point or interface or connector 34, for example, along edge 36 of opening 32′. Stent 30 and extension 35 may be connected at one or more open ends. Connector 34 may be any suitable interface that may secure the extension 35 to the stent 30. For example, in one embodiment, stent 30 may be made of plastic and extension 35 may be made of plastic thread or metal wire. In this exemplary embodiment, connector 34 may be a melted interface that connects stent 30 and extension 35. In another embodiment, stent 30 may be made of wire mesh and extension 35 may be made of for example metal wire. Other suitable materials may be used. Connector 34 may be for example a clasp or knot that connects stent 30 and extension 35. In other embodiments, the stent tube 38 and the extension may be of the same material, and may be formed of one continuous integral piece of material. In one embodiment a connector need not be used.

A stent according to embodiments of the present invention may be made of any suitable material, for example, plastic, silicone, a suitable polymer, metal, wire mesh and may take any suitable design including, for example, any suitable curvature, protrusions; length or diameter and may be inserted into any suitable lumen, for example, blood vessels or the pancreatic or bile ducts.

In some embodiments a protrusion or attachment (such as tip 14, bulge 24 or extension 35) may aid in securing a stent to a stent removal device, for example, forceps, a clasp or a hook or any other suitable device that may secure a stent for example at its tip. The stent may be removed, for example, by pulling or exerting force on the protrusion or attachment. The stent may be removed, for example, with forceps that may enter the body for example, via an endoscope. Other methods of removal may be used.

Securing or grabbing prior art stents may be difficult since there may not be a defined protrusion for the grasping device to clasp. The grasping device may clasp the side of the stent, which may cause the stent to fold or bend during removal, which may be harmful. A stent according to embodiments of the present invention may be grabbed or secured more easily at its tip than prior art stents. The stent may then be removed from the body, for example, by pulling. This may exert a force on the protrusion or extension, instead. of exerting a force along the body of the stent that may cause the stent to bend or fold during removal. A stent according to embodiments of the present invention may be secured and removed in a more desirable orientation and may be less likely to bend or fold during removal than a prior art stent.

Selecting which opening to modify may be irrelevant in the case where a stent is symmetric. However, a stent may have asymmetric features that may make the stent's specific orientations inside the body preferable. A stent according to some embodiments of the present invention may be designed so that its protrusion may be preferably oriented for stent removal.

Embodiments of the present invention include producing a stent, such as an embodiment of a stent described above or shown in FIGS. 1-3. In one embodiment, a stent may be cut at an opening to produce for example, a protruding tip. The stent may be made of, for example, plastic, or solid material. In one embodiment, a stent may be melted at a tip to produce for example a bulge.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

1. A stent, comprising:
a passageway; and
a plurality of openings,
wherein one opening of the plurality of openings includes a section of an edge that protrudes farther from a perpendicular cross-section of the stent than any other section of the edge,
wherein the perpendicular cross-section is that taken closest to the end of the one opening where the cross-section is fully contained by the stent material.

2. The stent of claim 1, wherein the passageway comprises solid material.

3. The stent of claim 1, comprising a bulge formed along the protruding edge.

4. The stent of claim 3, wherein the bulge along the protruding edge is formed by melting the protruding edge.

5. A stent, comprising a tube including at least two openings, wherein the tube is cut at one opening; and
the line of the cut is not perpendicular to the axis of the tube.

6. The stent of claim 5, wherein the passageway comprises solid material

7. The stent of claim 5, comprising a bulge formed along the protruding edge.

8. The stent of claim 7, wherein the bulge along the protruding edge is formed by melting the protruding edge.

9. A stent, comprising:
a tube; and
an extension connected to the tube,
wherein the extension and the tube are not part of one continuous piece of material.

10. The stent of claim 9, wherein the extension is attached to the stent at an opening.

11. The stent of claim 9, wherein the extension is attached to the stent at a protruding Lip.

12. The stent of claim 9, wherein the extension is attached to the stent by a connector.

13. A stent, comprising:
a plastic tube; and
an appendage attached to an end of the plastic tube.

14. The stent of claim 13, wherein the appendage is integral to the plastic tube.

15. The stent of claim 13, wherein the appendage is attached to the stent by a connector.

16. The stent of claim 13, wherein the appendage is a string.

17. The stent of claim 13, wherein the appendage is plastic.

18. A method, comprising cutting a stent at an opening to produce a protruding tip.

19. The method of claim 18, wherein the stent comprises plastic.

20. The method of claim 18, wherein the stent comprises solid material.

21. A method, comprising melting a tip of a stent to produce a bulge.

22. The method of claim 21, wherein the stent comprises plastic.

23. The method of claim 21, wherein the stent comprises solid material.
